(19) United States
(12) Patent Application Publication
(10) Pub. No.: US 2014/0357731 A1
(43) Pub. Date: Dec. 4, 2014

(54) HEALING CREAM

(71) Applicant: NOVO SOLUTIONS MD, LLC, Paradise Valley, AZ (US)

(72) Inventor: ALVIN DAVID NEEDLEMAN, Las Vegas, NV (US)

(21) Appl. No.: 14/294,570

(22) Filed: Jun. 3, 2014

Related U.S. Application Data
(60) Provisional application No. 61/830,169, filed on Jun. 3, 2013.

(51) Int. Cl. A61K 9/00; A61K 47/26; A61K 9/06; A61K 47/10

(52) U.S. Cl. CPC A61K 9/0014; A61K 47/10; A61K 47/26; A61K 9/06
USPC 514/785

(57) ABSTRACT

A method for formulating a cationic and non-ionic oil in water emulsion, preferably in a cream form, containing growth factors, preferably platelet derived growth factors, specific matrikine peptides, botanicals, emollients, nano-molecules, antioxidants, antiseptics, neuro-peptides, anti-dermatophytics, polysaccharides, sensorial peptides, osmotically driven moisturizers, and siloxane barriers. For application to the feet and other affected parts of the body for the relief of diabetic skin and tissue trauma.

### CROSS-REFERENCE TO RELATED APPLICATIONS

This application is based on and claims priority to U.S. Provisional Application Ser. No. 61/830,169, filed on Jun. 3, 2013, which is incorporated herein by reference in its entirety.

### STATEMENT REGARDING FEDERALLY SPONSORED RESEARCH OR DEVELOPMENT

Not applicable.

### REFERENCES CITED U.S. PATENT DOCUMENTS

60/986,560 November 2007, Hammock, et al

10/307,509 November 2002, Wolicki

6,271,196 August 2001, O'Brien

8,372,382 February 2012, Norman

### BACKGROUND OF THE INVENTION

The impact of type two diabetes on the human physiology is the result of a catastrophic series of biochemical, vascular and cellular events resulting in serious, sometimes irreversible consequences.

The development of the healing cream of the present invention takes into account the cellular, vascular, biochemical and tissue changes that take place and applies the technology to repair, reverse, and reconstruct the damage.

About 8% of Americans have diabetes. Unfortunately, this number is growing.
It is estimated that more than 25 million Americans are affected by diabetes.
Diabetic complications also involve the kidney's, eyes (retinopathy) and the extremeties.

### BRIEF SUMMARY OF THE INVENTION

The present invention is a spreadable readily absorbed healing cream that is site focused on the successful repair-restructure and enhancement of the physical, biological, and chemical properties of the three main skin layers, the sub dermal musculature and the basement membrane, and the Dermal Epidermal Junction (DEJ). One preferred embodiment of the healing cream of the present invention will be sold by NovoSolutions MD as a NovoSerum Formula.

The applications of the cream are for prophylactic, acute and chronic skin trauma inflicted surgically, accidentally, or as results of a disease state. The healing cream of the present invention is a robust growth factor driven formula for the relief, repair, and healing of general trauma induced wounds and diabetic wounds, decubitus ulcers, non-healing ulcers, venous stasis ulcers, incontinence skin breakdown, and pressure sores.

The healing cream of the present invention closes decubitus ulcers, non-healing ulcers, venous stasis ulcers, incontinence skin breakdown, pressure sores, softens hardened compressed skin, moisturizes and repairs dry skin, repairs cracked skin, prevents bacterial-fungal, and dermatophytic infection.

### DETAILED DESCRIPTION OF PREFERRED EMBODIMENT

The strategy of the invention is based upon the release of formula components for the relief of hardened tissue, compressed skin, cracked tissue, and ulcerated tissue. The following formula ingredients generate the energy for osmotic tissue moisturization, epithelialization over an introduced matrix, reduced peripheral wound tension, barrier gas exchange, and antiseptic and dermatophytic repair of traumatized tissue.

Cetearyl alcohol, polysorbate 60: non-ionic emulsifying wax containing a combination of the two emulsifiers polysorbate 60 & cetearyl alcohol. White waxy flakes, odorless. Water-insoluble, dispersible in water & oil. Saponification value 14.0 Max. pH Value: 5.5-7 (3% solution). CAS# 8005-44-5; 67762-27-0, 9005-67-8. Non-ionic oil-in-water emulsifier has high electrolyte tolerance, has also dispersing, thickening, and solubilizing properties (stabilizes essential oils).

Cetearyl alcohol and behentrimonium methosulfate in which the combination is an exceptionally effective cationic self-emulsifying wax and quaternary conditioner for skin care applications. As a behenyl derivative, its longer chain fatty moiety (C22) offers distinct formulating advantages over the more traditional lauryl- to stearyl-based quaternaries. Incroquat Behenyl TMS provides enhanced conditioning and emulsifying properties in combination with the greater mildness, substantive to the skin, outstanding conditioning agent, improved mildness/reduced irritation potential, primary emulsifier, thickening agent/stabilizer, emulsifies high levels of silicone, vegetable-derived a quaternary amine salt derived from the natural plant oil Colza commonly known as Rapeseed oil.

Butyrosperum parkii certified organic plant fat of the nuts of the African Karite tree (ambuk butter), 100% organic, contains allantoin, vitamins A and E, and many cinnamic and unsaponifiable lipids e.g., stearic and oleic acids for a potent moisturizing effect. A robust moisturizer and emollient, provides optimum skin care protection. Postulated to protect also in part from UV rays and shown to have anti-irritant & soothing properties.

Glycerin an effective humectant and moisturizer attracting water to the intercellular space of the skin, protects the lipids of skin cells, a good solvent for various water-insoluble ingredients with effective emollient and lubricant properties.

Myristamidopropyl PG-dimonium chloride phosphate a substantive to the skin, offering excellent conditioning properties and sensorial benefits. Provides remarkable antimicrobial and preservative boosting effects. Mild to skin and eyes, making it suitable for sensitive skin applications.

Phosphatidylcholine is the active ingredient in lecithin. Every cell membrane in the body requires phosphatidylcholine (“PC”). It is also a major source of the neurotransmitter acetylcholine. PC is considered an excellent water-binding agent and aids in the penetration of other ingredients into the skin. It absorbs well without feeling greasy or heavy. PC is an intermediate in the formation of liposomes.

PPG3-Benzyl ether myristate is easily emulsified, detackifies popular emollients, and offers low skin spreading properties. Silicone-like feel and functionality, improves emulsion esthetics, barrier and film forming.

Cyclopentasiloxane is a conditioner, delivery agent, lubricant, and solvent. Cyclopentasiloxane has a unique fluidity that makes it easily spreadable. When applied to the skin, it gives a silky and slippery feeling to the touch and has water repellent properties by forming a protective barrier on the skin.

Squalane or pure vegetally derived squalane is a natural hydrocarbon in cholesterol biosynthesis, derived from olive oil, highly resistant to oxidation, has a high affinity to skin cells due to its skin-identical structure. Present in human sebum at a level of about 10%.

A specialty emollient, high resistance to oxidation, high affinity to skin and its natural lipids, excellent skin-penetrating qualities, non-irritating, non-allergenic, hypo-comedogenic.

Triethanolamine is a pH adjuster, stabilizes emulsions and preservatives, foam stabilizer which improves efficacy of preservatives by stabilizing the pH value.

Triglyceride an exceptional low-viscosity emollient & lubricant leaving a soft and non-greasy feel, improves spreading excellent vehicle and solvent for lipophilic active ingredients.

Palmitoyl oligopeptide-palmitoyl tetrapeptide-7 stimulates the fibroblast cells and promotes the production of procollagen. Fibroblasts continue to create collagen even in old age, but their levels decrease as a person ages, causing dryness and tissue stiffness. The matrikines Pal-GQPR and Pal-GHK acts in synergy to repair dryness caused by age, activating the body's natural production of elastin, collagen, and hyaluronic acid. Collagen, the amino acids responsible for the skin's smoothness progressively gets lost as the individual ages. Collagen breaks down into smaller chains called peptides, which signals the skin to produce more collagen. The peptides are mimicked by making the body think that more collagen as well as elastin and hyaluronic acid are needed by the skin. One of the two active components of Palmitoyl oligopeptide Pal-GHK is a peptide believed to indicate the degradation of the matrix of the skin. When fibroblasts, the cells that produce the skin matrix detect higher levels of GHK, they synthesize more skin matrix assuming that they are being lost faster. Pal-GHK is a more advanced version of GHK because it penetrates the skin better so it can stimulate the replenishment of the skin matrix through topical application. The other active component is Palmitoyl tetrapeptide-7-Pal-GQPR is known to reduce the skin cells' production of interleukin-6-IL-6. The molecule known as IL-6 is known to promote inflammation and thus contribute to the skin matrix degradation. Resulting in the loss of skin elasticity and firmness, palmitoyl tetrapeptide-7 helps in stimulating the replenishment of the skin matrix and slows down its degradation.

Erythritol Homarine HCL protects against hyper- and hypotonic stress, desiccation and dehydration stress. Ensures aqua homeostasis of the skin cells and progressively rehydrates the different layers of the skin.

Acetyl hexapeptide-8 causes acetylcholine inhibition by elevating acetylcholinestrase.

Acetylcholinesterase, also known as AChE or acetylhydrolase, is a serine protease that hydrolyzes the neurotransmitter acetylcholine. AChE is found at mainly neuromuscular junctions and cholinergic brain synapses, where its activity serves to terminate synaptic transmission. It belongs to the carboxylesterase family of enzymes.

Analysis of the mechanism of action showed that Acetyl Hexapeptide-8 significantly inhibited neurotransmitter release. Inhibition of neurotransmitter release was due to the interference of the Hexapeptide with the formation and/or stability of the protein complex that is required to drive Ca²⁺-dependent exocytosis, cellular direction of secretory vesicles namely the vesicular fusion known as SNARE=soluble NSF attachment receptor complex.

L fucose, D-Galactose, galacturonic acid the combination provides a long-term osmotic binding of water and moisturizes the skin. It penetrates deeply into the epidermis, protecting it from water loss. Additionally, it creates a protective film on the skin's surface, providing protection against the transepidermal water loss called TEWL. It is absorbed by the skin in one hour and has an active duration of three hours and functions to relax peripheral wound tension.

Poly D-glucosamine or PDG properties allow it to rapidly clot blood, and have been shown in testing to quickly stop bleeding. PDG is hypoallergenic, and has natural antibacterial properties. PDG's properties also allow it to be used in transdermal ingredient delivery; it is mucoadhesive in nature, reactive, and most importantly, has a positive charge under acidic conditions. Capable of forming a matrix to accelerate epithelialization in difficult to heal cratered and non-healing ulcers and pressure sores.

L-proline is a non-essential amino acid which means the body can synthesize sufficient quantities to support its needs. L-proline is an essential component of collagen and because of its unique structure supports respiratory barrier formation and function.

L-arginine improves the health of the feet of diabetics suffering from the reduction of circulation. In the study presented by Dr. Eric T. Fossel and Dr. Steven A. Shaya they report that use of the L-Arginine in a cream, i.e., Novo Serum Formula, significantly improved both temperature and blood flow in the feet of the diabetic subjects they studies. Temperature increased 5° F. at the metatarsal and 8° F. at the big toe. Flow increased 33% at the metatarsal and 35% at the achilles.

Retinyl palmitate Microcaps is pure vitamin A or retinol and is based on a nano-delivery system. Vitamin A is entrapped in non-visible microcapsules made of a shell of a natural sugar complex carboxymethyl cellulose-chitosan. This capsule stabilizes retinol to avoid degradation reactions and maintain the activity of the ingredient.

NovoSerum® a composition containing multiple platelet derived growth factors and added, genetic receptor stimulating, matrikine peptides PAL-GQPR and PAL-GHK and over 350 molecules in solution including enzymes, hormones, antioxidants, antigens, vitamins, amino acids, proteins, and minerals. Prepared from umbilical cord blood obtained by FDA regulatory approved methods, pretested for microbial and fungal contamination. The mother's blood is pre-tested for infectious and viral contaminants as required for umbilical cord blood storage or for clinical utilization at accredited Cryobanking facilities according to F.A.C.T.

Multiple growth factors effectively initiate a robust reconstructive process causing a marked increase in the generation of procollagen with a subsequent increase in the precursors responsible for the replacement of lost collagen. An increase in Glycosaminoglycan's resulting in an increase in the addition of new cells to the extracellular matrix. These structural water binding elements of the skin rapidly help thicken and support the skin matrix and the cellular structure of the dermal sub layers. NovoSerum® growth factors reverse damage caused by a loss of cellular nutrients, loss of cellular communications, loss of collagen, loss of skin matrix density and a loss of the primary cellular moisture system. The development of NovoSerum® constitutes a proprietary method to extract and concentrate platelet derived growth factors from human umbilical cord blood, and activate them to begin the process of wound healing.

N-acetyl tyrosil arginyl hexedecyl ester promotes well-being by reducing the perception of unpleasant sensations. It modulates the perception of temperature, chemical aggressions, and tactile sensation. By putting the skin at ease, it creates a more comfortable, and relaxed local sensation allowing increased formula activity.

Hyaluronic acid biotechnologically produced from glucose, soy peptone, and yeast extract does not contain any animal products. Hyaluronic acid is a natural polysaccharide large sugar molecule that occurs in various tissues of the skin, synovial fluids of joints, and connective tissues. Responsible for tissue hydration, lubrication, and tissue stability, holds 500 times its own weight of water. This powerful moisturizer and humectant, provides smoothness and softening to the skin and promotes the growth of new skin cells.

Dimethyl isosorbide is a high purity natural alternative-solvent and transport carrier which offers a safe, effective, and enhanced delivery mechanism for active ingredients. In addition to improving performance, its use as an epidermal penetration enhancer can allow formulators to reduce the amount of actives required to achieve effects, resulting in lower skin irritation with more aggressive active ingredients.

Melaleuca alternafolia contains constituents called terpenoids, which are antiseptic, antifungal and anti dermatophytic. The compound terpinen-4-ol is the most abundant and is thought to be responsible for the antimicrobial activity.

Cyclopentasiloxane and dimethicone/vinyl dimethicone cross-polymer is a clear silicone elastomer dispersed in cyclopentasiloxane. The resulting product is a smooth, highly viscous clear gel which has a unique sensory characteristics and the ability to act as a thickening agent. It is responsible for barrier activity promoting a natural exchange of respiratory gases while enhancing moisture retention and providing excellent aesthetics, a smooth, silky, luxurious feel that is non-greasy and hydrophobic.

Phenoxyethanol sorbic acid, benzylalcohol dehydroacetic acid provides effective broad spectrum protection against gram-positive and gram-negative bacteria, yeast, and mold while imparting emolliency to the finished product.

Propanediol and Lecithin, increases ingredient percutaneous absorption, provides time-release potential and bioavailability by forming tissue penetrable-shaped bi-layer-lipid vesicles encapsulating formula actives.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

One aspect of the present invention is directed to a method for the use of charged coupled emulsifiers for the formation of a thixotropic matrix to support the transfer of active principles to the surface of the skin and into the epidermis.

Another aspect of the present invention is directed to a method for improving the absorption and transport of active materials using solvents and materials with muco-adhesive properties.

Another aspect of the present invention is directed to a method for the production of antioxidant skin penetration for lipid absorption.

Another aspect of the present invention is directed to a method of increasing spread ability, fluidity, water repellence, and a protective barrier on the skin surface.

Another aspect of the present invention is directed to a method for the protection of hyper and hypotonic tissue moisture stress, desiccation and dehydration stress.

Another aspect of the present invention is directed to a method for osmotically infusing a carbohydrate moisturizer into the skin.

Another aspect of the present invention is directed to a method for depositing of active materials at a specific site under the influence of polar charged components.

Another aspect of the present invention is directed to a method for softening hardened and compressed human tissue and relieving peripheral wound stress.

Another aspect of the present invention is directed to a method for removing surface bacterial contamination.

Another aspect of the present invention is directed to a method for preventing the growth of dematophyte instigated infections, fungus and inter-digital cracking, and peeling of the skin.

Another aspect of the present invention is directed to a method for repairing traumatized tissue by the wound healing effects generated by growth factors.

Another aspect of the present invention is directed to a method of introducing enzymatic antagonization of elements of a neurotransmitter.

Another aspect of the present invention is directed to a method of improving cellular signaling through matrikine peptide stimulation of genetic receptors.

Another aspect of the present invention is directed to a method of increasing vascular dilation with a corresponding increase in blood flow.

Another aspect of the present invention is directed to a method of introducing peptides to promote collagen production.

Another aspect of the present invention is directed to a method of introducing peptides to promote increase extracellular matrix density and moisture content.

Another aspect of the present invention is directed to a method of introducing an increase in the depth and speed of absorption of actives. Providing shaped bi-layer lipid vesicles for transport of aqueous actives through the stratum corneum.

1. What is claimed by the invention is a method for the preparation of charged coupled emulsifiers for the formation of a thixotropic matrix to support epithelialization and the transfer of active principles to the surface of the skin and into the epidermis.
